(19) United States
(12) Patent Application Publication
(10) Pub. No.: US 2007/0038142 A1
(43) Pub. Date: Feb. 15, 2007

(54) Method and device for delivering visual stimuli with head mounted display during vision training

(76) Inventors: David P. Todd, Boynton Beach, FL (US); Bernhard Sabel, Berlin (DE)

Correspondence Address: BROMBERG & SUNSTEIN LLP, 125 SUMMER STREET, BOSTON, MA, 02110-1618, US

(21) Appl. No.: 11/394,154

(22) Filed: Mar. 30, 2006

Related U.S. Application Data
(60) Provisional application No. 60/666,455, filed on Mar. 30, 2005.
(60) Provisional application No. 60/763,589, filed on Jan. 31, 2006.

(51) Int. Cl. A61B 13/00

(52) U.S. Cl. 600/558

(57) ABSTRACT

A head mounted display for diagnosing or training vision of a subject. In one embodiment, the head mounted display includes a first display that presents a first visual image to a first eye of the subject in an identified position of regard with respect to a specified zone in the field of view of the subject. This embodiment may also include a second display that presents a second visual image to a second eye of the subject in an identified position of regard with respect to a specified zone in the field of view of the subject. This embodiment may also include an anterior support section coupled to the first and second displays, the anterior support section orienting the display in front of the eyes of the subject and may also include a posterior support section coupled to the anterior support section.

### PRIORITY

The present application claims priority from U.S. Provisional Patent Application Ser. No. 60/666,455, filed Mar. 30, 2005 and from U.S. Provisional Patent Application Ser. No. 60/763,589, filed Jan. 31, 2006, both of which are hereby incorporated by reference in their entirety.

### TECHNICAL FIELD

The present invention relates to systems and methods to provide vision training to subjects including patients with vision impairment. In particular, the invention is directed toward delivering visual stimuli to a head mounted display worn by the subject as part of vision training.

### BACKGROUND ART

Therapeutic vision training is directed toward improving the visual performance of patients with vision impairments by stimulating their vision systems using visual stimuli. For example, as documented in U.S. Pat. No. 6,664,356 and U.S. application Ser. No. 10/503,869, each of which is hereby incorporated by reference herein, presenting visual stimuli to a transition zone and/or to areas of intact vision of a human's visual system, may allow improvement in the subject's vision. A “transition zone,” as used herein and in any appended claims, refers to an area of deteriorated vision or residual vision function, or partial visual system injury. An area of intact vision refers to an area in a subject's field of view which appears to be without visual deficits. Other applications of vision training are discussed below. Such training may be carried out on a personal computer for home use, the training performed in sessions on a daily schedule for a set period of time (e.g., an hour).

To identify the location and orientation of a subject's head relative to a display used to present visual stimuli and to properly stimulate the correct zones or areas in a subject's visual field, previous training regimens relied upon a subject fixing his or her gaze in a particular location. The natural tendency of persons to move after being in an unsupported, fixed position for a relatively long period of time may cause misalignment of the visual stimuli relative to a subject's visual field. Such misalignment may limit the effectiveness of a training session. Even if a subject attempts to fix his head's position relative to a display, identifying the proper position may be difficult, especially for individual subjects outside of a clinical setting. As well, the amount of time required to properly align the relative position of the display with a subject's visual field can be substantial.

### SUMMARY OF THE INVENTION

One embodiment of the present invention is directed to a head mounted display for diagnosing or training vision of a subject. The head mounted display of this embodiment includes means for forming a visual image according to a current characterization of an eye of the subject as well as means for displaying the visual image to an eye of the subject. The head mounted display of this embodiment also includes means for mounting the means for displaying in front of the eye of the subject. The means for mounting include sensors for tracking the location of the eye. The head mounted display of this embodiment also includes means for coupling the head mounted display to the head of the subject.

Another embodiment of the present invention is directed to a head mounted display for diagnosing or training vision of a subject. The head mounted display of this embodiment includes a first display that presents a first visual image to a first eye of the subject in an identified position with respect to a specified zone in the field of view of the subject. The head mounted display of this embodiment also includes a second display that presents a second visual image to a second eye of the subject in an identified position with respect to a specified zone in the field of view of the subject, wherein the first visual image is different than the second visual image. The head mounted display of this embodiment also includes an anterior support section coupled to the first and second displays, the anterior support section orienting the display in front of the eyes of the subject; and a posterior support section coupled to the anterior support section.

Another embodiment of the present invention is directed to a device for diagnosing or training vision of a subject. The device of this embodiment includes a head mounted display and a processor in communication with the display, the processor generating the visual stimuli presented by the display, the processor configured to generate the visual stimuli based upon a current characterization of the eye.

In another embodiment of the present invention a system for training the vision of a subject is disclosed. The system of this embodiment includes a display for presenting a visual image to an eye of the individual, the display allowing a field of view between 40 and 60 degrees. The system of this embodiment also includes an anterior support section for orienting the display in front of the eye of the subject and a posterior support section for positioning the head of the subject relative to the display. In this system, the vision of the subject is trained by presenting visual stimuli on the display.

Another embodiment of the present invention is directed to ahead mounted display for diagnosing or training vision of a subject having a head. The head mounted display of this embodiment includes a processor that forms a first visual image according to a current characterization of a first eye of the subject. The head mounted display of this embodiment also includes a first display that displays the first visual image to the first eye of the subject and a support portion that positions the first display in front of the first eye of the subject, the support portion including sensors for tracking the location of the first eye.

The present invention may used, for example, to train the transition zone and/or to areas of intact vision of a human's visual system. The present invention may also be used to train one or more blind regions of a human's visual system as is disclosed in U.S. Provisional Patent Application Ser. No. 60/763,589. In addition, the present invention may be used to treat retinal diseases as is disclosed in U.S. patent application Ser. No. 11/343,960, entitled “Methods for Treatment of Retinal Diseases,” filed Jan. 31, 2006, which is hereby incorporated by reference.

### BRIEF DESCRIPTION OF THE DRAWINGS

The foregoing features of the invention will be more readily understood by reference to the following detailed description, taken with reference to the accompanying drawings, in which:

FIG. 1 depicts a perspective view of a head mounted display in accordance with an embodiment of the invention.

FIGS. 2A-2C depict several embodiments of head mounted display in accordance with the present invention. FIG. 2A illustrates a virtual reality embodiment, FIG. 2B, a goggles embodiment, and FIG. 2C, an eyeglasses embodiment.

### DETAILED DESCRIPTION OF SPECIFIC EMBODIMENTS

Various embodiments of the invention described herein are directed toward devices and methods for training a subject to compensate for impairment of vision. Such embodiments may include the use of a head mounted display that positions the eye of a subject relative to a display and display image. The display presents visual stimuli that are viewed by the subject, the stimuli resulting in training of the impaired vision of the subject. A head-mounted display with side-pieces is referred to herein as “goggles.”

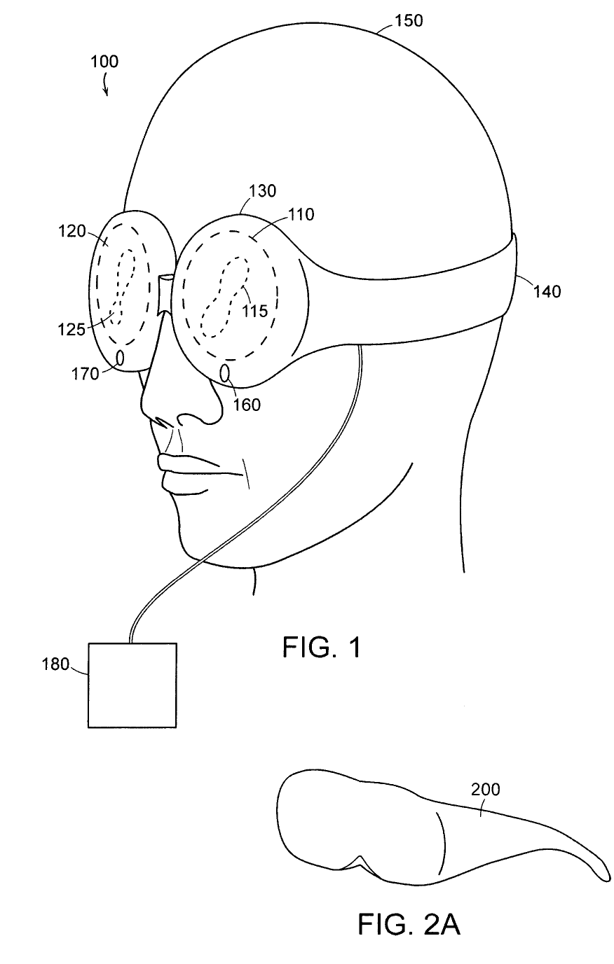

One particular embodiment of the invention is depicted in FIG. 1. A head mounted display 100 is shown having a left display 110, a right display 120, an anterior support section 130, and a posterior support section 140. The left display 110 presents a left visual image 115 to the left eye of the subject. The right display 120 presents a right visual image 125 to the right eye of the subject. The anterior support section 130 is coupled to the left display 110 and to the right display 120 and orients the left display 110 in front of the left eye of the subject and the right display 120 in front of the right eye of the subject. The posterior support section 140 is coupled to the anterior support section 130 and secures the head mounted display 100 to the head 150 of the subject, though other arrangements for securing the head mounted display 100 to the head 150 of the subject are within the scope of the present invention.

The head mounted display 100 presents the visual stimuli to the subject to impart the benefits of vision training through stimulating particular zones or areas of a subject's visual field. Zones of a subject's visual field that may be stimulated particularly include a transition zone, and/or an area of intact vision of a human's visual system, where the terms “transition zone” and “area of intact vision” have been defined above.

Techniques of vision training are discussed in U.S. Pat. No. 6,664,356 and U.S. application Ser. No. 10/503,869, the contents of which are hereby incorporated herein by reference. Such techniques of training, and corresponding diagnostics, typically maintain the head of a subject in relation to a display for an extended period of time. For example, during diagnostic testing of vision, a diagnostic session typically is conducted over approximately a two-hour period in three-repetitions, each repetition lasting approximately 30 minutes with breaks. Likewise, a therapeutic session to at least partly restore vision may last approximately 30 minutes, being conducted twice a day, six days a week. Thus, it is advantageous to utilize a head mounted display in order to reduce or eliminate the strain associated with maintaining a particular head position for an extended period of time. Further, a head mounted display may lessen the strain on the subject's eyes. Fixation is not forced. A subject's gaze is allowed to wander and images presented to the subject are adjusted to compensate for the current position of the subject's eyes. The current position of the eye may be monitored, for example, by an ASL series 6000 eye tracker produced by Advance Science Laboratories.

The head mounted display 100 automatically and correctly presents visual stimuli to the subject without requiring the subject to maintain a constant head position and fixation point and without the need for extensive calibration of the display. That is, the visual stimuli are based on current characterizations of the eyes. Current characterizations include positions of the subject's eyes, alignment of the subject's gaze, and known deficiencies in the subject's visual field. The head mounted display 100 detects the positions of the left and right eyes and their orientations. Since there is automatic adjustment for eyes, testing is a much more relaxed and comfortable experience for the subject. The therapeutic benefits of vision training may be easily obtained on a transportable unit that a subject may utilize at the home or at another desired location.

The head mounted display 100 reduces the possibility of error in adjusting a display or head-positioning device, which could result in sub-optimal vision training. Since the population of potential subjects of vision training includes a sizable fraction of persons with cognitive disabilities, the potentially increased ease-of-use and reliability associated with embodiments of the invention present attractive potential advantages over existing systems.

Returning to the embodiment of the invention shown in FIG. 1, the head mounted display 100 includes an anterior support section 130 which is preferably constructed of a lightweight, strong material capable of maintaining its shape and orientation with respect to the head of the subject in any head position. For example, the anterior support section 130 may be constructed of an acrylic or other lightweight polymer/composite material and may include an elastic material covering the area of the anterior support section 130 in contact with the head of the subject.

The posterior support section 140 is configured to stabilize the head 150 of a subject in a comfortable manner. The posterior support section 140 may include a head strap preferably constructed of an elastic material capable of maintaining the anterior support section 130 in contact against the forehead of the subject.

Though embodiments of the invention depicted in the Figures utilize a head strap as the posterior support section 140 of the head mounted display 100, other structures that stabilize the anterior support section 130 without such an element are also consistent with a head-support. Optionally, the posterior support section 140 may be a rigid material that is shaped with a curvature to accommodate attachment over the ears of the subject in the manner of eyeglasses.

In addition, the posterior support section may include a system suitable to excite the visual cortex while the vision stimulation is ongoing. A system suitable for this purpose would be Transcranial Magnetic Stimulation (TMS) which is normally used to treat depression, as disclosed in U.S. Pat. No. 6,926,660. The TMS stimulation may be synchronized with the vision stimulation to achieve optimum excitement of the neurons in the cortical region corresponding to the transition region, blind field or intact field, as determined by the therapy.

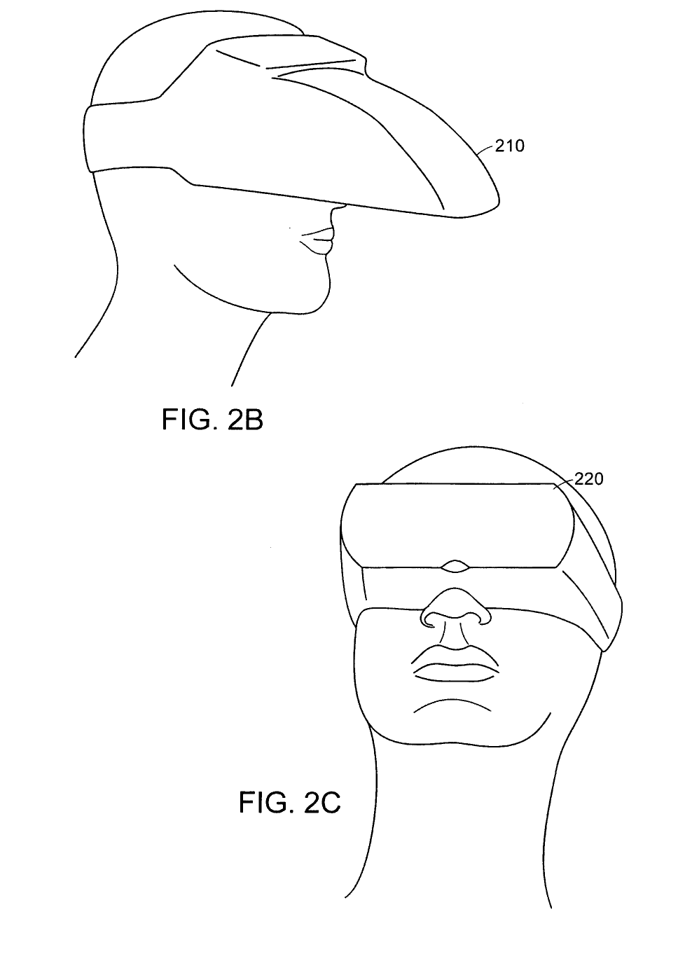

In one embodiment, the head mounted display 100 may be a set of goggles 200 as illustrated in FIG. 2A. The goggles 200 are preferably light-tight and display images to the left and right eyes of the subject from interior surfaces. In another embodiment, a head mounted display 100 may contain display apparatus 210 similar to that found in night vision enabling equipment or in virtual reality games, as illustrated in FIG. 2B. In another embodiment, the head mounted display may be a similar to a set of eyeglasses 220 as illustrated in FIG. 2C. In this embodiment, images are projected onto interior surfaces in front of the eyes. Regardless of the type of display used, in one embodiment, the head mounted display may include displays that allow for a field of view between 40 and 60 degrees. In one example this may be achieved by using curved displays as the display apparatus.

The head mounted display 100 is used to orient a display that presents visual stimuli to train the vision of the subject. The left display 120 is positioned in front of the left eye of the subject and the right display 130 is positioned in front of the right eye of the subject. The display images may be the result of self-contained illumination, as, for example, electroluminescence, or the result of passive illumination, as, for example, an LCD display illuminated by ambient illumination. Adjacent to the left display 130 is a left sensor 160 and adjacent to the right display 140 is a right sensor 170. Sensors 160 and 170 detect the angular positions of the left and right eyes, respectively. Eye position information is transmitted to a processor, such as CPU 180, which determines the positions and directions of regard of the left and the right eyes.

The CPU 180 generates the visual stimuli presented by the left 110 and right 120 displays. The generation of the visual stimuli depends in part upon the orientation and location of the left and right eyes, as detected by left and right sensors, 160 and 170 respectively. Since the orientations and locations of the eyes will dictate what portions of the display are in the visual field of the subject, such information allows the CPU 180 to generate visual stimuli in the appropriate portions of the left 110 and right 120 display to train the subject's vision. This may be of particular relevance in specifically training particular zones of a subject's visual field. In some embodiments, the images shown on the left and right displays 110 and 120 may be different from one another.

CPU 180 may take the form of a transportable computer, for example a personal computer or a dedicated computer. Images 115 and 125 in electrical form may be communicated from CPU 180 to head mounted display 100 by wire or in a wireless manner. If wireless, communication may be at radio frequencies (RF) and may use a communication protocol such as Bluetooth™.

With automatic calibration and continuous adjustment of the left 110 and right 120 displays, desired training may be delivered to the left and right eyes despite a lack of fixed positioning of the head and lack of a designated fixation point. As a result, CPU 180 may be configured to produce the same visual stimuli regardless of the nature of the gaze of the subject and without a need to calibrate the displays.

Head mounted displays, and methods of using head mounted displays, may be directed to non-therapeutic training applications. Head mounted displays may be especially useful where quick visual identification of targets is advantageous. Non-limiting examples include training the vision of athletes (e.g. golfers) to improve hand-eye coordination or reaction time, training in military applications (e.g., helping improve their capability and efficiency in identifying targets on screens of equipment), and training for airplane pilots.

Further embodiments of the invention are directed toward devices and methods that diagnose a condition of vision in a subject before training the vision of the subject. In one embodiment of the invention, CPU 180 may generate visual stimuli to be presented on left 110 and right 125 displays for use in diagnosing a condition of vision in a subject. The head mounted display may be particularly preconfigured to conduct this diagnostic task by presenting left 115 and right 125 images to perform the diagnostic task. The diagnostic performance may be used to generate the visual stimuli used to train a subject subsequently, in accord with other embodiments of the invention described herein. The corresponding methods including performing a diagnosis before conducting the training are within the scope of the present invention.

All aforementioned embodiments of the invention are intended to be merely exemplary and numerous variations and modifications will be apparent to those skilled in the art. All such variations and modifications are intended to be within the scope of the present invention as defined in the appended claims.

What is claimed is:

1. A head mounted display for diagnosing or training vision of a subject having a head, the head mounted display comprising:
means for forming a first visual image according to a current characterization of a first eye of the subject;
first means for displaying the first visual image to the first eye of the subject;
means for mounting the first means for displaying in front of the first eye of the subject, the means for mounting including sensors for tracking the location of the first eye; and
means for coupling the head mounted display to the head of the subject.

2. A head mounted display according to claim 1, further including means for displaying a second visual image to a second eye of the subject.

3. A head mounted display according to claim 2, wherein the first visual image and the second visual image are different.

4. A head mounted display for diagnosing or training vision of a subject, the head mounted display comprising:
a first display that presents a first visual image to a first eye of the subject in an identified position with respect to a specified zone in the field of view of the subject;
a second display that presents a second visual image to a second eye of the subject in an identified position with respect to a specified zone in the field of view of the subject, wherein the first visual image is different than the second visual image;
an anterior support section coupled to the first and second displays, the anterior support section orienting the display in front of the eyes of the subject; and
a posterior support section coupled to the anterior support section.

5. A head mounted display according to claim 4, wherein the visual image is configured to present a selected range of fixation points to the subject.

6. A head mounted display according to claim 4, wherein the posterior support section includes a head strap.

7. A head mounted display according to claim 6, wherein the posterior support section includes a system suitable for exiting the subjects visual cortex.

8. A head mounted display according to claim 4, wherein head mounted display is configured to present optical stimuli to a zone within the intact visual field of the subject, to a zone outside the intact visual field of the subject, and a blind zone of the subject, and to train vision in one or more of the zones.

9. A device for diagnosing or training vision of a subject, the device comprising:
a head mounted display including sensors for tracking the location of at least one eye of the subject; and
a processor in communication with the display, the processor generating the visual stimuli presented by the display, the processor configured to generate the visual stimuli based upon a current characterization of the eye.

10. A device according to claim 9, wherein the processor is configured to generate the visual stimuli based upon a plurality of current characterizations of the eye.

11. A device according to claim 10, wherein the processor is configured to generate the visual stimuli for diagnosing a visual condition of the subject.

12. A device according to claim 10, wherein the processor is configured to generate the visual stimuli for training the vision of the subject.

13. A system for training the vision of a subject comprising:
a display for presenting a visual image to an eye of the individual, the display allowing a field of view between 40 and 60 degrees;
an anterior support section for orienting the display in front of the eye of the subject; and
a posterior support section for positioning the head of the relative to the display;
wherein the training in performed by presenting visual stimuli on the display.

14. A head mounted display for diagnosing or training vision of a subject having a head, the head mounted display comprising:
a processor that forms a first visual image according to a current characterization of a first eye of the subject;
a first display that displays the first visual image to the first eye of the subject;
a support portion that positions the first display in front of the first eye of the subject, the support portion including sensors for tracking the location of the first eye.

15. A head mounted display according to claim 1, further including a second display that displays a second visual image to a second eye of the subject.

16. A head mounted display according to claim 15, wherein the first visual image and the second visual image are different.
